(19) United States
(12) Patent Application Publication
(10) Pub. No.: US 2026/0199703 A1
(43) Pub. Date: Jul. 16, 2026

(54) CRYSTALLINE LENS PROTECTOR FOR RADIATION THERAPY

(71) Applicants: PAPRICA LAB. CO., LTD., Seoul (KR); J AND YL CO., LTD., Gyeonggi-do (KR)

(72) Inventors: Jung In KIM, Seoul (KR); Sang Hoon HEO, Seoul (KR); Song Yi HEO, Seoul (KR); Min Jae CHOI, Seoul (KR); Sung Jun LIM, Gyeonggi-do (KR)

(73) Assignees: PAPRICA LAB. CO., LTD., Seoul (KR); J AND YL CO., LTD., Gyeonggi-do (KR)

(21) Appl. No.: 19/134,197

(22) PCT Filed: Mar. 15, 2024
(86) PCT No.: PCT/KR2024/003275

§ 371 Date: May 29, 2025

(30) Foreign Application Priority Data
Mar. 28, 2023 (KR) 10-2023-0040227

(51) Int. Cl. A61N 5/10; A61F 9/04; G21F 1/08

(52) U.S. Cl. CPC A61N 5/10; A61F 9/045; G21F 1/085; A61N 2005/1094

(57) ABSTRACT

The present invention relates to a crystalline lens protector for a radiation therapy, comprising: a shielding member attached to the front portion of the eyeball including a crystalline lens, so as to block radiation emitted at the eyeball from a radiotherapy machine; and a cover member provided on the outer side surface of the shielding member so as to block radiation backscattered to the eyelid from the shielding member. Therefore, the crystalline lens protector for a radiation therapy, according to the present invention, prevents eyelid exposure caused by the backscattering of radiation.

### TECHNICAL FIELD

The present disclosure relates to a crystalline lens protector for radiation therapy, and more particularly, to a crystalline lens protector for radiation therapy, which is capable of preventing lenticular exposure to radiation when radiation is used to treat an eyeball or a tumor near the eyeball.

### BACKGROUND ART

Ocular radiation therapy is used to treat tumors that arise in the ocular adnexa such as uveal melanoma and lymphoma. Especially, for the treatment of conjunctival lymphoma, the treatment targets to be irradiated are conjunctival fornix, palpebral conjunctiva, and bulbar conjunctiva, where the irradiation of these areas inevitably irradiates a lens. The lens is an ocular organ that is very sensitive to radiation, and may cause cataracts (lenticular opacity) even with a small amount of radiation (absorbed dose) of about 1.5 Gy. In particular, since the degree of risk increases with higher radiation dose, it is necessary to shield the lens against radiation to minimize cataracts.

For this reason, ocular radiation therapy is performed by installing a metal lens protector (hereinafter, referred to as “protector”) capable of blocking radiation on an eyeball's front surface, thereby preventing the radiation exposure of the lens.

The protector may be typically in the form of a roughly thin hemispherical vessel to cover the lens and its surrounding area, and has a rod-shaped handle on the surface thereof to make it easy for users to easily handle the protector.

This conventional protector is able to block the radiation emitted to the lens, but it may cause unnecessary additional radiation exposure of an eyelid by irradiating the eyelid that covers the protector with backscattering (a phenomenon in which radiation or atomic particles are scattered at an angle of 90 degrees or more to the angle of incidence) of radiation from the outer surface of the protector.

The conventional protector also has a handle in the shape of a circular rod, which protrudes upright from the center of the outer surface of the protector. Hence, the eyelid (upper eyelid) may be caught by the handle and not close completely, and the protector may not be installed in position (on the front surface of the eyeball including the lens) due to the force applied to the protector by the handle. In addition, the protector may be detached from the eyeball in some cases (if the patient is treated while standing) because the eyelid does not stably cover the protector.

The foregoing is intended for technical information possessed for derivation of the present disclosure or acquired in the process of derivation thereof by the inventor, which is not necessarily a known technique disclosed to the general public prior to the filing of the invention.

### CITATION LIST

(Patent Document 1) Korean Patent Application Publication No. 10-2022-0138986 (Oct. 14, 2022)

### DISCLOSURE

### Technical Problem

The present disclosure has been made in view of the above-mentioned problems, and an object of the present disclosure is to provide a crystalline lens protector for radiation therapy, which is capable of preventing unnecessary additional radiation exposure of eyelids by suppressing backscattering of radiation.

In addition, an object of the present disclosure is to provide a crystalline lens protector for radiation therapy, which enables eyelids to be completely closed normally, resulting in stable installation of the protector in position.

The present disclosure is not limited to the above-mentioned objects, and other objects of the present disclosure will be clearly understood by those skilled in the art to which the present disclosure pertains from the following description.

### Technical Solution

In accordance with an aspect of the present disclosure, there is provided a crystalline lens protector for radiation therapy, which includes a shield member attached to a front surface of an eyeball including a lens to shield the eyeball against radiation from a radiotherapy machine, and a cover member installed on an outer surface of the shield member to block radiation that is backscattered from the shield member to an eyelid.

The shield member may have a coupling protrusion formed in the center of the outer surface thereof, the cover member may have a coupling groove formed in the center of its inner surface, and the coupling protrusion may have a male thread formed on its outer peripheral surface and the coupling groove may have a female thread formed on its inner peripheral surface so that the shield member is screwed to the cover member.

The crystalline lens protector may further include an O-ring made of elastic material on an outer periphery of the bottom of the coupling protrusion.

The O-ring may be, at its inner periphery, inserted into and pressed against an undercut groove formed on the outer periphery of the bottom of the coupling protrusion.

The O-ring may be compressed between the shield member and the cover member.

The O-ring may be configured such that its bottom is pressed against a flat surface formed around the coupling protrusion of the shield member and its top is pressed against a portion around the entrance of the coupling groove of the cover member.

The cover member may have a handle formed on its outer surface, and the handle may be formed to protrude eccentrically and obliquely with respect to the center of the cover member.

The handle may be in the form of a curved plate in which its widthwise middle part is convex outwardly relative to both ends thereof.

The handle may have strap holes formed on both corners of the top thereof for insertion of respective fixing straps thereinto.

The shield member may include 89 to 90 wt % of tungsten (W), 6.5 to 6.8 wt % of nickel (Ni), 2.7 to 2.9 wt % of iron (Fe), 0.1 to 0.2 wt % of copper (Cu), and 0.15 to 0.2 wt % of molybdenum (Mo).

### Advantageous Effects

As described above, the crystalline lens protector for radiation therapy according to the present disclosure can prevent unnecessary additional radiation exposure of eyelids by suppressing backscattering of radiation.

In addition, the crystalline lens protector for radiation therapy according to the present disclosure enables the eyelids to be completely closed normally, resulting in the stable installation of the protector in position.

The present disclosure is not limited to the above-mentioned effects, and other effects of the present disclosure will be clearly understood by those skilled in the art to which the present disclosure pertains from the following description.

### BRIEF DESCRIPTION OF DRAWINGS

FIG. 1 is a perspective view illustrating a crystalline lens protector for radiation therapy (hereinafter, referred to as “protector”) according to an embodiment of the present disclosure.

FIG. 2 is a bottom perspective view of FIG. 1.

FIG. 3 is a perspective view illustrating a shield member, which is one component of the protector illustrated in FIG. 1.

FIG. 4 is a perspective view illustrating a cover member, which is another component of the protector illustrated in FIG. 1.

FIG. 5 is a front view of FIG. 1.

FIG. 6 is a right side view of FIG. 1.

FIG. 7 is a top view of FIG. 1.

FIG. 8 is a cross-sectional view taken along line A-A of FIG. 7.

FIG. 9 is a view illustrating a state of use of the protector illustrated in FIG. 1.

### MODE FOR DISCLOSURE

[List of Reference Numerals]

| | |
|---|---|
| 10: shield member | 10A: inner surface |
| 11: coupling protrusion | 12: undercut groove |
| 13: flat surface | 20: cover member |
| 21: coupling groove | 22: handle |
| 23: strap hole | 30: O-ring |
| 41: upper eyelid | 42: lower eyelid |
| 50: strap | 60: tape |

The accompanying drawings in the present disclosure may have been exaggerated for differentiation and clarity from the prior art and for the sake of understanding the technology. In addition, the terms used in the specification are terms defined in consideration of functions of the present disclosure, and these terms may change depending on the intention or practice of a user or an operator. Therefore, these terms should be defined based on the overall disclosures set forth herein. Meanwhile, the following embodiments are merely for the purpose of describing the components set forth in the appended claims and are not intended to limit the spirit and scope of the disclosure.

Throughout the specification, it will be understood that, when a component is referred to as “comprising” or “including” any component, it does not exclude other components, but can further comprise or include the other components unless otherwise specified.

In addition, it will be understood that, when a component is referred to as being “connected”, “joined”, or “coupled” to another component, it can be “directly connected”, “directly joined”, or “directly coupled” to the other component or it can be “indirectly connected”, “indirectly joined”, or “indirectly coupled” to the other component with other components being interposed therebetween. On the other hand, it will be understood that, when a component is referred to as being “directly connected”, “directly joined”, or “directly coupled” to another component, no intervening components are present.

In addition, when directional terms such as “before”, “after”, “up”, “down”, “left”, “right”, “one end”, “other end”, and both ends” are used, these terms should not be construed as limiting as they are used by way of example in relation to the orientation in the drawings disclosed herein. As used herein, the terms such as “first” and “second” should not be construed as limiting terms for distinguishing each component.

In order to more clearly describe features of embodiments of the present disclosure, a detailed description of matters widely known to those skilled in the art to which the following embodiments pertain will be omitted. In addition, a detailed description of parts irrelevant to the embodiment and description in the drawings will be omitted.

Hereinafter, exemplary embodiments of the present disclosure will be described in detail with reference to the accompanying drawings.

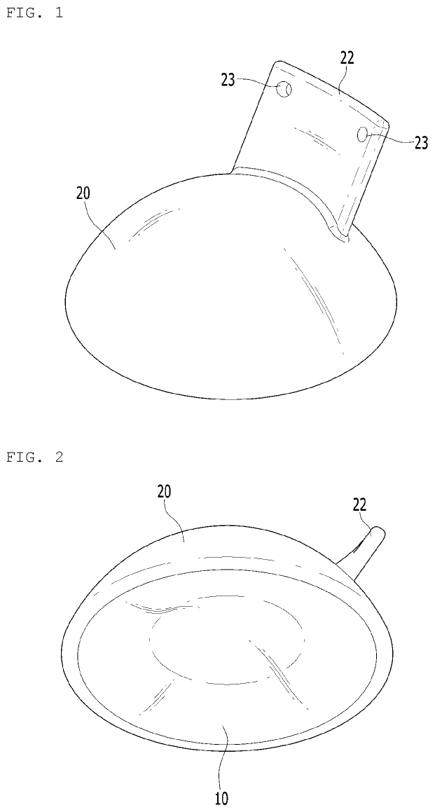

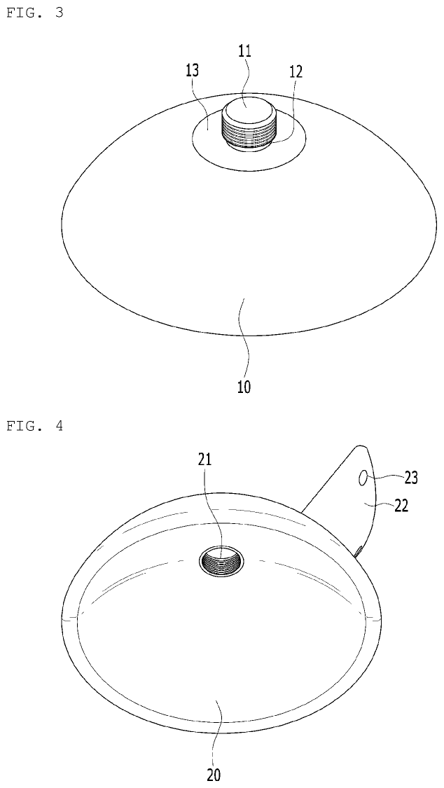

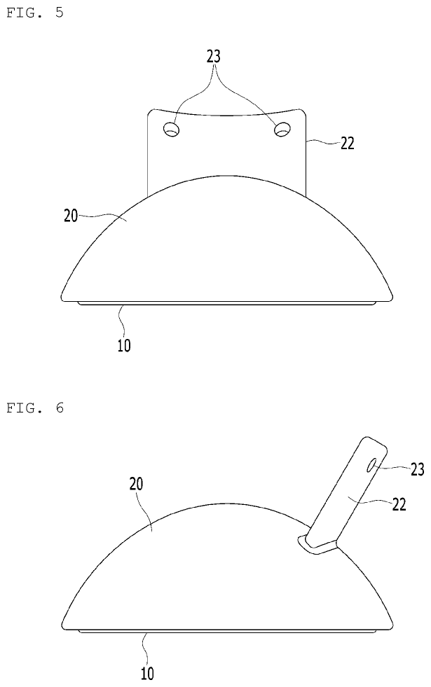

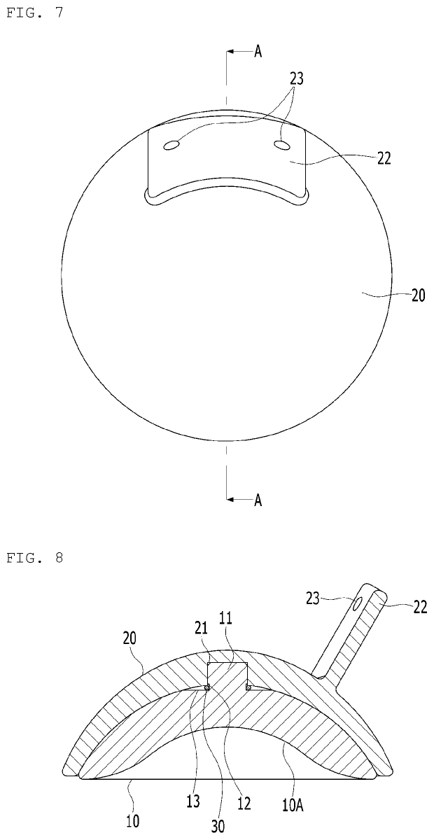

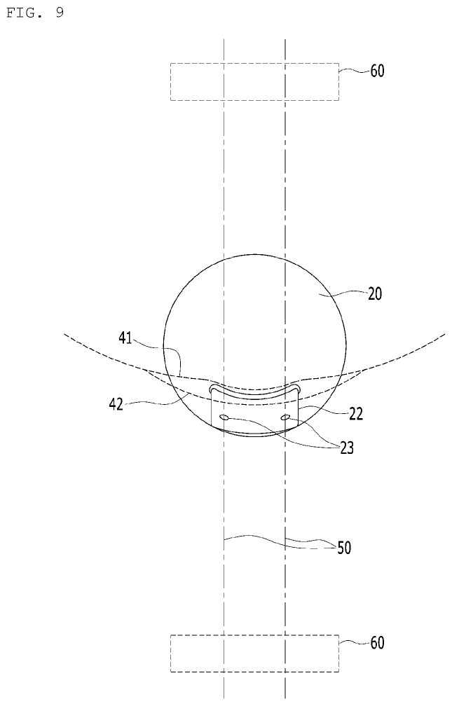

FIG. 1 is a perspective view illustrating a crystalline lens protector for radiation therapy (hereinafter, referred to as “protector”) according to an embodiment of the present disclosure. FIG. 2 is a bottom perspective view of FIG. 1. FIG. 3 is a perspective view illustrating a shield member, which is one component of the protector illustrated in FIG. 1. FIG. 4 is a perspective view illustrating a cover member, which is another component of the protector illustrated in FIG. 1. FIG. 5 is a front view of FIG. 1. FIG. 6 is a right side view of FIG. 1. FIG. 7 is a top view of FIG. 1. FIG. 8 is a cross-sectional view taken along line A-A of FIG. 7. FIG. 9 is a view illustrating a state of use of the protector illustrated in FIG. 1.

Referring to FIGS. 1 to 9, the crystalline lens protector for radiation therapy (hereinafter, referred to as “protector”) according to the embodiment of the present disclosure includes a shield member 10 and a cover member 20.

The shield member 10 is a component attached to the front surface of an eyeball including a lens to shield the eyeball against radiation from a radiotherapy machine.

The shield member 10 is a sintered product with tungsten (W) as a main raw material. Tungsten (W), together with lead (Pb), is a material with excellent radiation shielding effects and is suitable as a material for radiation therapy protectors since it is harmless to human bodies.

The shield member 10 is in the form of a roughly thin hemispherical vessel capable of covering the front surface of the eyeball. The shield member 10 has a smooth surface that is safe to come into direct contact with the eyeball and an eyelid (its medial area). During actual treatment, the shield member 10 is applied with ointment on the surface thereof and attached to the eyeball, thereby preventing corneal damage.

The shield member 10 may have a spherical inner surface 10A (see FIG. 8) having the same curvature as the front surface of the eyeball for stable close contact with the eyeball.

The shield member 10 has a coupling protrusion 11 formed in the center of the outer surface thereof. The coupling protrusion 11 is configured for coupling the shield member 10 and the cover member 20.

The coupling protrusion 11 is in the form of a circular column with a male thread on its outer peripheral surface.

The cover member 20 is a component installed on the outer surface of the shield member 10 to block the radiation that is backscattered from the shield member 10 to the eyelid.

The cover member 20 basically has the same shape (roughly hemispherical vessel shape) as the shield member 10 and is sized to cover the entire outer surface of the shield member 10. The cover member 20 has a spherical inner surface having the same curvature as the outer surface of the shield member 10, so that when the cover member 20 is coupled to the shield member 10, the inner surface of the cover member 20 is exactly in surface contact with the outer surface of the shield member 10.

The cover member 20 has lower radiation shielding power than the shield member 10, but is made of a material that has shielding power sufficient to block the radiation backscattered from the shield member 10. In this case, the material needs to be harmless to human bodies. To satisfy these conditions, the cover member 20 may be made of, for example, aluminum (Al).

The cover member 20 may be made of aluminum (Al) to effectively block the radiation backscattered from the shield member 10, thereby preventing unnecessary additional exposure of the eyelid to the radiation backscattered from the protector. Here, it is confirmed that there is no need to worry about the amount of radiation exposure of the eyelid since, from among the radiation incident on the protector, the radiation backscattered from the cover member 20 has a much weaker energy intensity than that backscattered from the shield member 10.

The cover member 20 has a coupling groove 21 formed in the center of the inner surface thereof. The coupling groove 21 is configured for coupling the shield member 10 and the cover member 20.

The coupling groove 21 is in the form of a circular groove, which corresponds to the coupling protrusion 11 of the shield member 10 and has a female thread formed on the inner peripheral surface thereof.

Accordingly, the coupling protrusion 11 and the coupling groove 21 allow the shield member 10 to be screwed to the cover member 20. In other words, the coupling protrusion 11 of the shield member 10 may be inserted and screwed into the coupling groove 21 of the cover member 20, thereby allowing the shield member 10 and the cover member 20 to be firmly coupled to each other. In this case, the female and male threads may be formed in interchangeable positions, which would be a substitution within an equal range.

As described above, the shield member 10 is made of tungsten (W) (melting point: 3415° C.) and the cover member 20 is made of aluminum (Al) (melting point: 660° C.), so it is difficult to bond them by fusion because they have significantly different melting points. Accordingly, the present disclosure solves this technical problem through a screwing structure.

However, when the coupling protrusion 11 (i.e., the male thread made of tungsten) of the shield member 10 is fastened to the coupling groove 21 (i.e., the female thread made of aluminum) of the cover member 20, metal dust (mainly aluminum dust) may be generated due to the friction between different metal materials. Given that the protector is used by attachment to the eyeball, which is a very sensitive body organ, this generation of metal dust may be a fatal defect factor of the product.

In addition, there is always a possibility that the screwing structure may cause disengagement due to the decrease in fastening force by the action of unexpected external force or over time.

In consideration of this, the protector may be provided with an O-ring 30 made of elastic material on the outer periphery of the bottom of the coupling protrusion 11, as illustrated in FIG. 8. In other words, the O-ring 30 may be provided at the screwing portion between the shield member 10 and the cover member 20.

An undercut groove 12 is formed on the outer periphery of the bottom of the coupling protrusion 11 of the shield member 10, so that the inner periphery of the O-ring 30 is inserted into the undercut groove 12. The undercut groove 12 is formed around the outer circumference of the coupling protrusion 11. The O-ring 30, which is fitted from the top of the coupling protrusion 11, may be stably maintained in installation position by insertion at its inner periphery into the undercut groove 12 when reaching the bottom of the coupling protrusion 11. In other words, the inner periphery of the O-ring 30 is inserted into and pressed against the undercut groove 12 formed on the outer periphery of the bottom of the coupling protrusion 11.

Thus, when the shield member 10 is assembled to the cover member 20 while the O-ring 30 is first fitted into the coupling protrusion 11 of the shield member 10, it is possible to prevent the delay of assembly therebetween due to the detachment of the O-ring 30 from the coupling protrusion 11.

In order to stably support the bottom of the O-ring 30, a flat surface 13 is formed around the coupling protrusion 11 on the upper surface of the shield member 10. The flat surface 13 forms the same plane as the bottom surface of the undercut groove 12. Thus, the O-ring 30, which is inserted at its inner periphery into the undercut groove 12, may be stably supported at its bottom on the flat surface 13. The flat surface 13 may be machined during entering of a tool in a direction perpendicular to the central axis of the coupling protrusion 11 to form the undercut groove 12 in the coupling protrusion 11. In this way, the bottom surface of the undercut groove 12 and the flat surface 13 may form the same plane.

When the coupling protrusion 11 is screwed to the coupling groove 21 while the O-ring 30 is installed in the undercut groove 12, the O-ring 30 is compressed between the shield member 10 and the cover member 20. As described above, the inner peripheral end of the O-ring 30 is inserted into the undercut groove 12 and is pressed against the inner wall thereof. In addition, the O-ring 30 is pressed at its bottom against the flat surface 13 formed around the coupling protrusion 11 of the shield member 10, and is pressed at its top against a portion around the entrance of the coupling groove 21 of the cover member 20 (against the inner surface of the cover member 20).

Thus, the above structure allows the path from the inside to the outside of the coupling groove 21 to be completely blocked by the O-ring 30, and may prevent, even though dust is generated by the friction between different metals when the coupling protrusion 11 is screwed to the coupling groove 21, the generated dust from flowing out of the coupling groove 21. Therefore, it is possible to eliminate a phenomenon in which metal powder (dust) generated from the protector damages the eyeball or the eyelid.

In addition, when the coupling protrusion 11 has been completely screwed to the coupling groove 21 (the outer surface of the shield member 10 is in close contact with the inner surface of the cover member 20), the O-ring 30 is compressed by a predetermined amount between the shield member 10 and the cover member 20 so that an elastic restoring force is applied in a direction parallel to the central axis of the coupling protrusion 11. This elastic restoring force acts as an adhesion force between the threads of the coupling protrusion 11 and the coupling groove 21, thereby increasing a screwing force. Thus, it is possible to prevent the detachment between the shield member 10 and the cover member 20 due to unexpected release of the screwing therebetween.

In other words, the O-ring 30 is installed between the shield member 10 and the cover member 20 to act as a seal ring between the two members, thereby preventing dust from leaking to the outside, and also acts as a tension spring between the two members, thereby helping to prevent wear due to excessive torque during screwing and to improve coupling force by increasing the friction between threads in the state of coupling between the two members.

Meanwhile, the cover member 20 has a handle 22 formed on the outer surface thereof, and the handle 22 protrudes eccentrically and obliquely with respect to the center of the cover member 20. In other words, the handle 22 is formed to be spaced outwardly from the center of the cover member 20 by a predetermined distance (to be roughly positioned at the middle portion between the center and the outer periphery of the cover member 20) and to protrude obliquely at a predetermined angle (approximately 30° to) 45° with respect to the central axis of the protector (the central axis of the coupling protrusion 11).

The handle 22 is in the form of a curved plate in which its widthwise middle part is convex outwardly relative to both ends thereof. In other words, the handle 22 has a curved upper surface (facing the center of the cover member 20) that is concave toward the outside of the cover member 20, and the upper surface of the handle 22 has a roughly similar curvature to the grip surface of the user's thumb or index finger gripping the handle 22.

In addition, the handle 22 is formed so as to exist within the area range of the cover member 20 when viewed from above as in FIGS. 5 to 8. In other words, the top of the handle 22 does not extend radially outwardly from the cover member 20 but exists within the outer periphery thereof.

As described above, the handle 22 is formed to protrude obliquely at an eccentric position outwardly from the center of the cover member 20, which prevents interference between an upper eyelid 41 and the handle 22 when the protector is installed on the front surface of the eyeball. In other words, as illustrated in FIG. 9, the upper eyelid 41 moves completely toward a lower eyelid 42, allowing the eye to be closed normally.

Therefore, since the handle 22 is not pushed by the upper eyelid 41 so that the protector is installed in position on the front surface of the eyeball, it is possible to reliably shield the lens against radiation during treatment.

In addition, since the eye is closed completely so that the protector is fully covered by the eyelid except for the handle 22 and adjacent parts on both ends thereof, it is possible to prevent detachment of the protector from the eyeball during surgery. This is especially useful when the patient is treated while standing.

Moreover, since the handle 22 is in the form of a curved plate, it is possible to improve grip stability when the user grips the handle 22 with their thumb and index finger. In other words, it is possible to prevent rotation of the handle 22 when the user grips the handle 22 with their fingers as in the conventional case (in which the handle is in the form of a circular rod). Therefore, when the user handles the protector while gripping the handle 22, it is possible to prevent the protector from spinning and accurately adjust the posture of the protector as intended, and thus to attach the protector to the front surface of the eyeball more accurately and easily. In this case, since the surface of the handle 22 has a similar curvature to the grip surface of the user's finger, the user can feel a more comfortable sense of grip.

In addition, the handle 22 is curved as described above so as to correspond to the shape of the closed eyelid, and the curved shape of the upper and lower surfaces of the handle 22 is the same as the shape in which the respective ends of the upper eyelid 41 and the lower eyelid 42 are rounded downwards when the eye is closed.

Therefore, this curved shape of the handle 22 helps to minimize interference between the upper eyelid 41 and the handle 22 during installation of the protector and to install the protector in position and maintain the installed position thereof. It also helps to close the eye completely, allowing the eyelid to cover the entire protector and thus preventing detachment of the protector from the eyeball more reliably.

In addition, since the top of the handle 22 is within the area range of the cover member 20, there is no unnecessary radiation shielding area. If the top of the handle 22 is out of the area range of the cover member 20, the radiation shielding area by the handle 22 will be present on the outside of the cover member 20, namely, on the outside of the protector. In this case, if a tumor is in an area of the eyeball corresponding to the radiation shielding area by the handle 22, it becomes impossible to treat the tumor because that area may not be irradiated. Therefore, the above structure in which the top of the handle 22 exists within the area range of the cover member 20 is advantageous in preventing unnecessary radiation shielding areas from occurring.

The handle 22 has strap holes 23 formed on both corners of the top thereof for insertion of respective fixing straps 50 thereinto. In other words, the circular strap holes 23 may be formed through both corners of the top of the handle 22. The strap holes 23 are used to secure the protector more reliably using the straps 50 (medical threads) as illustrated in FIG. 9. The user may pass the straps 50 through the strap holes 23 and fix both ends of each strap 50 to the respective forehead and cheek of the patient to be treated with tapes 60 so as to apply a force to the protector toward the eyeball by the tension of the straps 50, thereby securing the protector.

In this case, there is an effect of preventing rotation of the handle 22 by spacing the two strap holes 23 from each other by a predetermined distance to install the two straps 50 at a distance from each other. Therefore, it is possible to prevent the protector from rotating and moving out of its original position during treatment while the protector is installed on the eyeball. In other words, it is possible to maintain the position of the protector more stably during treatment by forming the strap holes 23 on both sides of the top of the handle 22.

Meanwhile, the shield member 10 is manufactured by sintering and machining tungsten (W), nickel (Ni), iron (Fe), copper (Cu), and molybdenum (Mo) powders.

The shield member 10 contains 89 to 90 wt % of tungsten (W), 6.5 to 6.8 wt % of nickel (Ni), 2.7 to 2.9 wt % of iron (Fe), 0.1 to 0.2 wt % of copper (Cu), and 0.15 to 0.2 wt % of molybdenum (Mo), and has a composition ratio based on 100 wt % of the total of the components.

The shield member 10 manufactured with the above components and composition ratio may have a density of 17.6 g/cm³, which thus exhibits excellent radiation shielding performance.

Tungsten (W) is a main component of the material (alloy) for manufacturing the shield member 10 and for implementing high density and providing excellent shielding performance. If tungsten (W) is contained in an amount less than 89 wt %, the intended high density value as mentioned above may not be achieved. On the other hand, if it is contained in an amount in excess of 90 wt %, the sintering strength of the raw materials decreases.

Nickel (Ni) is a component that improves the high-temperature strength, corrosion resistance, and toughness of the alloy, and acts as a binder (binding agent) for the components included in the alloy. If nickel (Ni) is contained in an amount less than 6.5 wt %, the bonding between raw materials does not occur normally, which reduces the sintering strength of the alloy. On the other hand, if it is contained in an amount in excess of 6.8 wt %, the high density of the alloy may not be implemented.

Iron (Fe) is a component for improving the ductility and machinability of the alloy. If iron (Fe) is contained in an amount less than 2.7 wt %, the ductility and machinability of the alloy are insufficient, making it difficult to process the material into the shape of a shield member 10 through machining. On the other hand, if it is contained in an amount in excess of 2.9 wt %, the ductility of the alloy becomes excessive, which reduces the thread rigidity of the coupling protrusion 11 formed on the shield member 10.

Copper (Cu) is a component for reducing the possibility of bubbles when alloying elements are combined and for improving machinability. If copper (Cu) is contained in an amount less than 0.1 wt %, bubbles are generated between raw materials, which reduces the mechanical strength of the product and makes it impossible to achieve the high density mentioned above, resulting in a reduction in radiation shielding performance. On the other hand, if it is contained in an amount in excess of 0.2 wt %, this does not enhance the bubble suppression effect and acts as an obstacle to implementing the high density of the product.

Molybdenum (Mo) is a component for improving strength and deformation resistance at a certain temperature or more. If molybdenum (Mo) is contained in an amount less than 0.15 wt %, the desired level of strength and deformation resistance may not be obtained. On the other hand, if it is contained in an amount in excess of 0.2 wt %, this does not enhance the strength and deformation resistance anymore and acts as an obstacle to implementing high density of the product.

Meanwhile, the process of manufacturing the shield member 10 is as follows.

First, raw materials (in the form of powder) are mixed to satisfy the above composition ratio. The raw materials are then mixed homogeneously by a powder mixer.

Next, the mixture of powder is fed into a press machine and compressed and molded under cold or hot conditions. At this stage, a short circular rod-shaped material is formed.

Next, the material (compressed and molded product) is fed into a vacuum electric furnace and pre-sintered at a temperature of approximately 300 to 500° C. to provide a homogeneous particle distribution and shape retention performance.

Next, the pre-sintered product is fed into a furnace and sintered at a temperature of approximately 1100 to 1300° C. In this case, as a binder material (nickel) melts, the particles of tungsten are densely bonded to each other, increasing the density of the material (achieving density).

Next, the sintered product is treated by heating at a temperature of approximately 600° C. to improve mechanical properties such as strength, durability, and ductility, and to eliminate internal stress.

Next, the internal cracks, density, hardness, elasticity, etc. of the material are tested to exclude defective products and select normal products.

The material (intermediate product in the form of a rod) manufactured by the above sintering process is machined into the shape and dimension of the shield member 10 using a computerized numerical control (CNC) machine. At this time, the overall shape of the shield member 10, namely, the roughly thin hemispherical vessel shape, and the shape of the coupling protrusion 11 on the outer surface thereof are outlined.

Next, in the state in which the outer surface of the product, whose shape and size have been machined, is fixed by a vacuum adsorption fixture, the inner surface of the product is polished with an abrasive to form a smooth surface that is safe to come into direct contact with the eyeball.

Next, a thread is formed on the outer peripheral surface of the coupling protrusion 11 for coupling the cover member 20 to the polished product.

Meanwhile, the cover member 20 is manufactured by machining aluminum material using a 5-axis machine. In this case, both the outer surface and inner surface of the cover member 20 including the handle 22 may be machined using a single machine.

After the shape and dimension of the cover member 20 have been machined, the outer surface of the cover member 20 is sanded to ensure a surface roughness that is good enough to come into contact with the eyelid, and anodizing is performed on the outer surface of the cover member 20 or the entire surface of the product.

The implementation of anodizing can prevent corrosion by forming an oxide film on the surface of the cover member 20. In addition, the implementation of anodizing can provide psychological stability to patients by imparting various colors to the surface of the cover member 20 to avoid the uniform color (generally, gray color) of medical supplies.

The shield member 10 and the cover member 20 manufactured as described above are then washed with medical alcohol.

Next, the O-ring 30 is assembled into the undercut groove 12 of the shield member 10, and the coupling protrusion 11 is screwed to the coupling groove 21 to couple the shield member 10 and the cover member 20, thereby completing the assembly of the protector.

As described above, the crystalline lens protector for radiation therapy according to the present disclosure can suppress backscattering of radiation to prevent unnecessary additional radiation exposure of the eyelids and enables the eyelids to be completely closed normally, resulting in the stable installation of the protector in position.

While the present disclosure has been described with respect to the embodiments illustrated in the drawings, it will be obvious to those skilled in the art that such embodiments are provided by way of example only. It will be understood by those skilled in the art that various modifications and other equivalent embodiments may be made without departing from the spirit and scope of the disclosure as defined in the following claims. Therefore, the true technical protection scope of the present disclosure should be defined by technical concepts of the appended claims.

### INDUSTRIAL APPLICABILITY

The present disclosure relates to a crystalline lens protector for radiation therapy, and is applicable to industrial fields related to manufacture of radiation-resistive protectors or shields that require suppression and reduction of backscattering of radiation.

1. A crystalline lens protector for radiation therapy, comprising:
a shield member attached to a front surface of an eyeball comprising a lens to shield the eyeball against radiation from a radiotherapy machine; and
a cover member installed on an outer surface of the shield member to block radiation that is backscattered from the shield member to an eyelid.

2. The crystalline lens protector according to claim 1, wherein:
the shield member has a coupling protrusion formed in the center of the outer surface thereof;
the cover member has a coupling groove formed in the center of its inner surface; and
the coupling protrusion has a male thread formed on its outer peripheral surface and the coupling groove has a female thread formed on its inner peripheral surface so that the shield member is screwed to the cover member.

3. The crystalline lens protector according to claim 2, comprising an O-ring made of elastic material on an outer periphery of the bottom of the coupling protrusion.

4. The crystalline lens protector according to claim 3, wherein the O-ring is, at its inner periphery, inserted into and pressed against an undercut groove formed on the outer periphery of the bottom of the coupling protrusion.

5. The crystalline lens protector according to claim 4, wherein the O-ring is compressed between the shield member and the cover member.

6. The crystalline lens protector according to claim 5, wherein the O-ring is configured such that its bottom is pressed against a flat surface formed around the coupling protrusion of the shield member and its top is pressed against a portion around the entrance of the coupling groove of the cover member.

7. The crystalline lens protector according to claim 1, wherein the cover member has a handle formed on its outer surface, and the handle is formed to protrude eccentrically and obliquely with respect to the center of the cover member.

8. The crystalline lens protector according to claim 7, wherein the handle is in the form of a curved plate in which its widthwise middle part is convex outwardly relative to both ends thereof.

9. The crystalline lens protector according to claim 8, wherein the handle has strap holes formed on both corners of the top thereof for insertion of respective fixing straps thereinto.

10. The crystalline lens protector according to claim 1, wherein the shield member comprises 89 to 90 wt % of tungsten (W), 6.5 to 6.8 wt % of nickel (Ni), 2.7 to 2.9 wt % of iron (Fe), 0.1 to 0.2 wt % of copper (Cu), and 0.15 to 0.2 wt % of molybdenum (Mo).
